(19) United States
(12) Patent Application Publication
(10) Pub. No.: US 2023/0298715 A1
(43) Pub. Date: Sep. 21, 2023

(54) Information Processing Apparatus, Information Processing Method, and Program

(71) Applicant: TERUMO KABUSHIKI KAISHA, TOKYO (JP)

(72) Inventors: Masaru IKEGAMI, Fujisawa-shi (JP); Tomoko UEMURA, Hadano-shi (JP); Yoshihito MACHIDA, Sagamihara-shi (JP)

(73) Assignee: TERUMO KABUSHIKI KAISHA, Tokyo (JP)

(21) Appl. No.: 18/168,802

(22) Filed: Feb. 14, 2023

(30) Foreign Application Priority Data
Mar. 15, 2022 (JP) 2022-040567

(51) Int. Cl. G16H 10/60; G16H 20/10

(52) U.S. Cl. CPC G16H 10/60; G16H 20/10

(57) ABSTRACT

A non-transitory computer-readable medium, an information processing apparatus, and an information processing method that causes a computer to execute a process that includes: acquiring order information including patient identification information for identifying a patient registered in a medical information system, date information indicating a date on which a medication has been prescribed to the patient, and medication identification information for identifying the medication prescribed to the patient; acquiring medication administration record information indicating a record of a medication administered to the patient by using one or more of the patient identification information, the date information, and the medication identification information; adding data included in the acquired medication administration record information to the order information to generate combination information; and outputting the generated combination information.

### CROSS-REFERENCES TO RELATED APPLICATIONS

This application claims priority to Japanese Patent Application No. 2022-040567 filed on Mar. 15, 2022, the entire content of which is incorporated herein by reference.

### TECHNOLOGICAL FIELD

The present disclosure generally relates to an information processing apparatus, an information processing method, and a program.

### BACKGROUND DISCUSSION

Japanese Patent Application Publication No. 2007-11711 A describes a technology related to a medical information system configured to assist a medical practice.

In a medical information system, information regarding medications prescribed by doctors to patients, vital data of patients who have been treated, and the like are generally recorded. Therefore, it is conceivable to generate a data set for learning including a set of information such as the prescribed medications and information such as the vital data (e.g., data associated with vital signs) of the patients, perform machine learning using the data set for learning, and generate a trained model. When such a trained model is used, it is possible to predict a change in vital information by using information such as a medication and information such as vital data as inputs. Such a trained model can be used, for example, to predict prognoses of patients and to assist doctors in planning treatment plans.

However, in the conventional configuration, information regarding medications administered to patients are not sufficient, and there is room for improvement in performing the prediction of prognoses of patients, statistical analysis on treatment contents, and the like with relatively high accuracy.

### SUMMARY

An information processing apparatus, an information processing method, and a program are disclosed which enable acquisition of more detailed information regarding a medication administered to a patient.

A non-transitory computer-readable medium storing a computer program executed by a computer processor that executes a process according to an embodiment of the present disclosure that includes: acquiring order information including patient identification information for identifying a patient registered in a medical information system, date information indicating a date on which a medication has been prescribed to the patient, and medication identification information for identifying the medication prescribed to the patient; acquiring medication administration record information indicating a record of a medication actually administered to the patient by using one or more of the patient identification information, the date information, and the medication identification information; adding data included in the acquired medication administration record information to the order information to generate combination information; and outputting the generated combination information.

As an embodiment, one or more pieces of the order information prescribed by a prescription slip (i.e., an identical prescription slip) are acquired. The identical prescription slip having order information that includes the same order information as another prescription slip for the patient. For each of the one or more pieces of order information, the medication administration record information is acquired using one or more of the patient identification information, the date information, and the medication identification information included in the order information. Information obtained by adding data of the medication administration record information acquired for each of the one or more pieces of order information to each of the one or more pieces of order information is generated as the combination information.

As an embodiment, the medication administration record information on the patient identical to the patient identified by the patient identification information of the order information is acquired.

As an embodiment, the medication administration record information in which administration of the medication identical to the medication identified by the medication identification information of the order information that has been recorded is acquired.

As an embodiment, the medication administration record information in which administration has been recorded during a period until a predetermined number of days elapses since the date indicated by the date information of the order information is acquired.

As an embodiment, the order information further including administration method information indicating an administration method of the medication is acquired, and the medication administration record information in which administration by an administration method identical to the administration method indicated by the administration method information of the order information is recorded is acquired.

As an embodiment, information including a medication administration time that is time at which the medication has been administered is acquired as the medication administration record information, and the medication administration time is added as the data to the order information to generate the combination information.

As an embodiment, information including start and end times which are a start time and an end time of administration of the medication is acquired as the medication administration record information, and the start and end times are added as the data to the order information to generate the combination information.

As an embodiment, information including a dose of the medication is acquired as the medication administration record information, and the dose is added as the data to the order information to generate the combination information.

An information processing apparatus according to an embodiment of the present disclosure includes a control unit configured to: acquire order information including patient identification information for identifying a patient registered in a medical information system, date information indicating a date on which a medication has been prescribed to the patient, and medication identification information for identifying the medication prescribed to the patient; acquire medication administration record information indicating a record of a medication actually administered to the patient by using one or more of the patient identification information, the date information, and the medication identification information; add data included in the acquired medication administration record information to the order information to generate combination information; and output the generated combination information.

An information processing method according to an embodiment of the present disclosure is an information processing method executed by a computer, and includes: acquiring order information including patient identification information for identifying a patient registered in a medical information system, date information indicating a date on which a medication has been prescribed to the patient, and medication identification information for identifying the medication prescribed to the patient; acquiring medication administration record information indicating a record of a medication actually administered to the patient by using one or more of the patient identification information, the date information, and the medication identification information; adding data included in the acquired medication administration record information to the order information to generate combination information; and outputting the generated combination information.

According to an embodiment of the present disclosure, it is possible to acquire more detailed information regarding a medication administered to a patient.

### BRIEF DESCRIPTION OF THE DRAWINGS

FIG. 1 is a diagram illustrating a functional configuration example of a medical information system according to an embodiment of the present disclosure.

FIG. 2 is a diagram illustrating a hardware configuration example of an information processing apparatus of FIG. 1.

FIG. 3 is a flowchart illustrating an example of processing contents executed by the information processing apparatus of FIG. 1.

FIG. 4 is a view illustrating an example of order information recorded in an order information database (DB) of FIG. 1.

FIG. 5 is a view illustrating an example of medication administration record information recorded in a medication administration record information database (DB) of FIG. 1.

FIG. 6 is a view illustrating an example of estimating medication administration record information corresponding to order information.

FIG. 7 is a view illustrating an example of combination information generated on the basis of the order information of FIG. 4 and the medication administration record information of FIG. 5.

### DETAILED DESCRIPTION

Set forth below with reference to the accompanying drawings is a detailed description of embodiments of an information processing apparatus, an information processing method, and a program. Note that since embodiments described below are preferred specific examples of the present disclosure, although various technically preferable limitations are given, the scope of the present disclosure is not limited to the embodiments unless otherwise specified in the following descriptions. In the drawings, parts having the same configuration or function are denoted by the same reference sign. In the description of the present embodiment, redundant description of the same part is sometimes omitted or simplified as appropriate.

Medical Information System

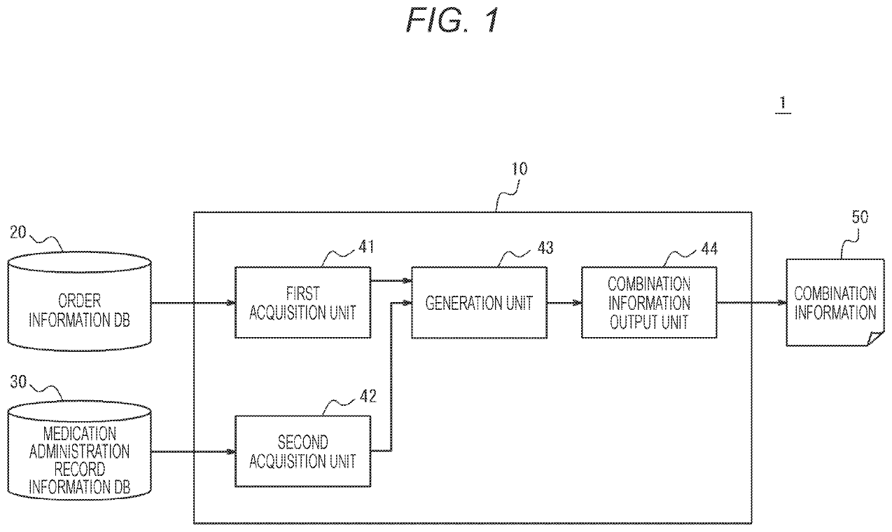

FIG. 1 is a diagram illustrating a functional configuration example of a medical information system 1 according to the embodiment of the present disclosure. As illustrated in FIG. 1, the medical information system 1 can include an information processing apparatus 10, an order information database (DB) 20, and a medication administration record information database (DB) 30. The information processing apparatus 10 is connected to the order information DB 20 and the medication administration record information DB 30 via a network to enable mutual communication.

The order information DB 20 is a database (DB) recording order information that is information regarding a medication prescribed by a doctor to a patient. The order information may include, for example, patient identification information for identifying a patient registered in the medical information system 1, date information indicating a date on which a medication has been prescribed to the patient, and medication identification information for identifying the medication prescribed to the patient, but may include information other than these.

The medication administration record information DB 30 is a database (DB) recording medication administration record information that is information indicating a record of a medication actually administered to a patient. The medication administration record information DB 30 may include, for example, a medication administration time that is time when a medication has been administered, start and end times which are a start time and an end time of the administration of the medication, and a dose of the medication, but may include information other than these.

The information processing apparatus 10 generates and outputs combination information 50 based on the order information recorded in the order information DB 20 and the medication administration record information recorded in the medication administration record information DB 30. The information processing apparatus 10 can be achieved by, for example, a computer such as a personal computer (PC), a workstation (WS), or a tablet, but is not limited to these apparatuses.

The information processing apparatus 10 can include functional elements of a first acquisition unit 41, a second acquisition unit 42, a generation unit 43, and a combination information output unit 44. The first acquisition unit 41 acquires the order information from the order information DB 20. The second acquisition unit 42 uses one or more of the patient identification information, the date information, and the medication identification information included in the order information acquired by the first acquisition unit 41 to acquire the medication administration record information indicating the record of the medication actually administered to the patient from the medication administration record information DB 30. The generation unit 43 adds data, included in the medication administration record information acquired by the second acquisition unit 42, to the order information acquired by the first acquisition unit 41 to generate the combination information 50. The combination information output unit 44 outputs the combination information 50 generated by the generation unit 43.

In this manner, the information processing apparatus 10 generates and outputs the combination information 50 by adding the data included in the medication administration record information corresponding to the order information to the order information. The combination information 50 includes information regarding a medication administered to a patient, which is included in only one of the order information and the medication administration record information. That is, the combination information 50 has more detailed information regarding the medication administered to the patient than each single piece of information of order information and medication administration record information acquired by an existing medical information system. Therefore, the information processing apparatus 10 enables acquisition of the more detailed information regarding the medication administered to the patient in the existing medical information system. When a trained model is generated by generating a data set for learning including a set of the combination information 50 and information such as vital data of the patient and performing machine learning using this data set for learning, it is possible to predict a prognosis of the patient with relatively higher accuracy. Furthermore, it is possible to acquire information with relatively higher usefulness or reliability by statistically analyzing the combination information 50.

The vital data used in the machine learning may be, for example, data (data related to vital information or vital signs of a patient) indicating a time-series change in a heart rate, a body temperature, a respiratory rate, a systolic blood pressure, a diastolic blood pressure, a consciousness level, the amount of urine, an inhaled oxygen concentration, a blood oxygen saturation, a urinary oxygen saturation, and the like. Furthermore, in the machine learning, data such as basic information (for example, age, sex, height, and the like), treatment contents during hospitalization (for example, intubation, mechanical respiratory support, and the like), and test information (for example, the amount of urine, creatinine, CI, sodium, Ca, and the like) may also be used in addition to such vital data of a patient.

The present embodiment is described with an example in which the information processing apparatus 10, the order information DB 20, and the medication administration record information DB 30 are achieved by different apparatuses, but is not limited to such a configuration. For example, one or more of the order information DB 20 and the medication administration record information DB 30 may be achieved by the information processing apparatus 10. In addition, one or more of the information processing apparatus 10, the order information DB 20, and the medication administration record information DB 30 may be achieved by a plurality of apparatuses.

Information Processing Apparatus

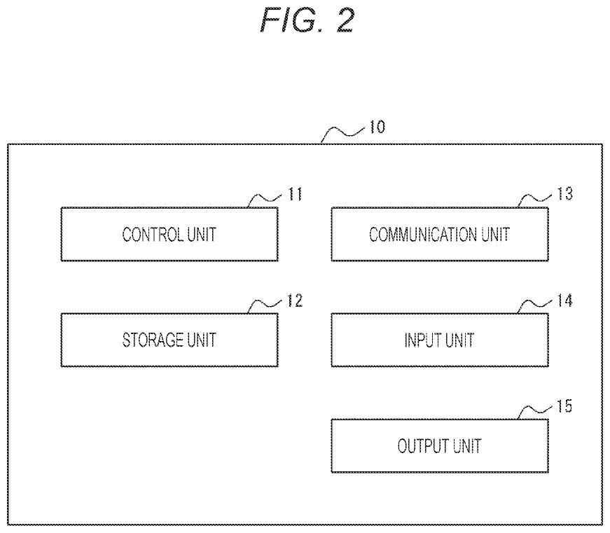

FIG. 2 is a diagram illustrating a hardware configuration example of the information processing apparatus 10 of FIG. 1. The information processing apparatus 10 can include a control unit 11, a storage unit 12, a communication unit 13, an input unit 14, and an output unit 15. Each of the order information DB 20 and the medication administration record information DB 30 may include a control unit, a storage unit, a communication unit, an input unit, and an output unit similarly to the information processing apparatus 10.

The control unit 11 can include at least one processor. The processor can include a general-purpose processor such as a central processing unit (CPU) or a dedicated processor specialized for specific processing. The control unit 11 may include an application specific integrated circuit (ASIC), a digital signal processor (DSP), a programmable logic device (PLD), a field-programmable gate array (FPGA), or any combination of the application specific integrated circuit (ASIC), the digital signal processor (DSP), the programable logic device (PLD) and the field-programmable gate array (FPGA). The control unit 11 may include a memory built in the processor or a memory independent of the processor. The control unit 11 executes processing related to the operation of the information processing apparatus 10 while controlling the respective units of the information processing apparatus 10.

The storage unit 12 can include at least one semiconductor memory, at least one magnetic memory, at least one optical memory, or any combination of at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 12 functions as, for example, a main storage device, an auxiliary storage device, or a cache memory. The storage unit 12 can store data to be used for the operation of the information processing apparatus 10 and data obtained by the operation of the information processing apparatus 10.

The communication unit 13 includes at least one communication interface. The communication interface can be, for example, a LAN interface, an interface compatible with a mobile communication standard such as the long term evolution (LTE), the fourth generation (4G) standard, or the fifth generation (5G) standard, or an interface compatible with a near field communication standard such as Bluetooth®. The communication unit 13 receives data to be used for the operation of the information processing apparatus 10 and transmits data obtained by the operation of the information processing apparatus 10. The information processing apparatus 10 can acquire the order information and the medication administration record information via the communication unit 13.

The input unit 14 includes at least one input interface. The input interface can be, for example, a physical key, a capacitive key, a pointing device, a touch screen provided integrally with a display, an imaging device such as a camera, or a microphone. The input unit 14 receives an operation of a user of inputting data to be used for the operation of the information processing apparatus 10. The input unit 14 may be connected to the information processing apparatus 10 as an external input device instead of being provided in the information processing apparatus 10.

The output unit 15 includes at least one output interface. The output interface can be, for example, a display or a speaker. The display can be, for example, a liquid crystal display (LCD) or an organic electro luminescence (EL) display. The output unit 15 outputs data obtained by the operation of the information processing apparatus 10. The output unit 15 may be connected to the information processing apparatus 10 as an external output device instead of being provided in the information processing apparatus 10.

Functions of the information processing apparatus 10 can be achieved by executing a program according to the present embodiment by a processor serving as the control unit 11. That is, the respective functional elements of the information processing apparatus 10, such as the first acquisition unit 41, the second acquisition unit 42, the generation unit 43, and the combination information output unit 44 described with reference to FIG. 1, can be implemented by software. The program causes a computer to execute the operation of the information processing apparatus 10 to cause the computer to function as the information processing apparatus 10. That is, the computer functions as the information processing apparatus 10 by executing the operation of the information processing apparatus 10 according to the program.

The program can be stored in a non-transitory computer-readable medium. The non-transitory computer-readable medium can be, for example, a flash memory, a magnetic recording device, an optical disk, a magneto-optical recording medium, or a ROM. The program can be distributed by, for example, selling, transferring, or lending a portable medium such as a secure digital (SD) card, a digital versatile disc (DVD), or a compact disc read only memory (CD-ROM) storing the program. The program may be distributed by storing the program in a storage of a server and transferring the program from the server to another computer. The program may be provided as a program product.

The computer temporarily stores, for example, the program stored in the portable medium, or the program transferred from the server in a main storage device. Then, the computer reads the program stored in the main storage device by the processor, and executes processing according to the read program by the processor. The computer may read the program directly from the portable medium and execute the processing according to the program. The computer may sequentially execute the processing according to the received program each time the program is transferred from the server to the computer. The processing may be executed by a so-called application service provider (ASP) type service that implements functions only by an execution instruction and result acquisition without transferring the program from the server to the computer. The program includes information that is provided for processing by an electronic computer and is equivalent to the program. For example, data that is not a direct command to the computer but has a property that defines processing of the computer corresponds to the “information equivalent to the program”.

Some or all of the functions of the information processing apparatus 10 may be implemented by a programmable circuit or a dedicated circuit as the control unit 11. That is, some or all of the functions of the information processing apparatus 10 may be implemented by hardware. For example, the first acquisition unit 41, the second acquisition unit 42, the generation unit 43, and the combination information output unit 44 described with reference to FIG. 1 may be implemented respectively by separate information processing apparatuses.

Information Processing Method

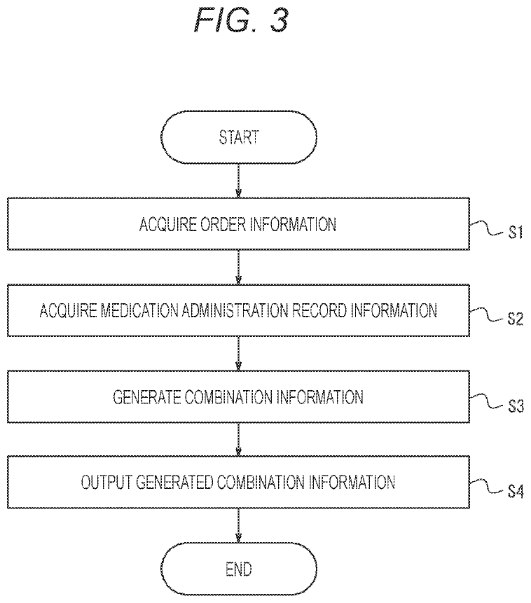

FIG. 3 is a flowchart illustrating an example of processing contents executed by the information processing apparatus 10 of FIG. 1. The operation of the information processing apparatus 10 described with reference to FIG. 3 can correspond to one of information processing methods. An operation of each step in FIG. 3 can be executed on the basis of control by the control unit 11 of the information processing apparatus 10.

In S1, the control unit 11 acquires order information. Specifically, the control unit 11 acquires the order information from the order information DB 20 via the communication unit 13. The order information may be, for example, information recorded as receipt data in an electronic medical record so as to claim for a medical treatment fee.

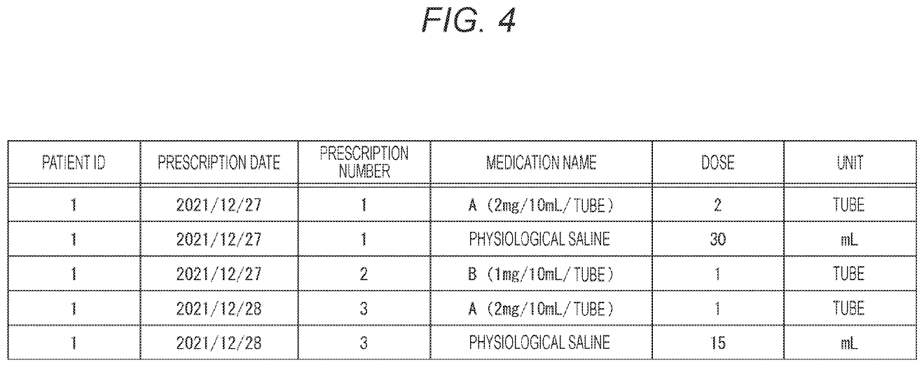

FIG. 4 is a view illustrating an example of the order information recorded in the order information DB 20 of FIG. 1. In FIG. 4, a “patient ID” is an example of patient identification information for identifying a patient registered in the medical information system 1. In the example of FIG. 4, the patient identification information represented by a number (“1”) may be text information such as a patient name. Furthermore, the “patient ID” may be information associated with not only a name of the patient but also a date of birth, identification information of a medical facility, information for identifying hospitalization in a unified facility, and the like.

A “prescription date” is an example of date information indicating a date on which a medication has been prescribed to the patient. A “prescription number” is information for identifying prescription given by a doctor. In the “prescription number”, the same number may be assigned to the same prescription slip, for example. In the example of FIG. 4, for the patient “1”, prescription is given twice (prescription numbers “1” and “2”) on Dec. 27, 2021. In the prescription identified with the prescription number “1”, “A (2 mg/10 mL/tube)” and “physiological saline” (i.e., saline) are prescribed. In the prescription identified with the prescription number “2”, “B (1 mg/10 mL/tube)” is prescribed. In the “prescription number”, numbers increasing in a time-series manner may be assigned to the same patient.

A “medication name” is a name of the medication prescribed to the patient, and is an example of medication identification information for identifying a medication prescribed to a patient. In the example of FIG. 4, the “medication name” can include information indicating an active ingredient amount, a dose of the medication, a unit, and the like, in addition to the name of the medication. For example, in the example of FIG. 4, “B (1 mg/10 ml/tube)” is prescribed in the prescription with the prescription number “2”. Here, “B” is information indicating a name of a medication. “1 mg” is information indicating an active ingredient amount of a medication. “10 ml” is information indicating a dose of the medication. The “tube” is information indicating a unit of the medication, for example, a container containing 1 mg of the active ingredient and a 10 ml dose of the medication. In the example of FIG. 4, a medication is identified using a name of the medication, but the medication identification information may be a numerical value for identifying a medication, a Japanese standard commodity classification number, a drug code listed in the National Health Insurance Drug Price Standard, a YJ code, another character string, or the like.

A “dose” is a dose of the medication prescribed to the patient. A “unit” is a unit of the “dose”. For example, in prescription identified with a prescription number “3”, “1 tube” of “A (2 mg/10 mL/tube)” is prescribed, and “15 ml” of “physiological saline” (or saline) is prescribed.

The order information may include information other than the information illustrated in FIG. 4. For example, the order information may further include administration method information indicating an administration method of the medication, description of items such as “usage”, and information such as the number of prescription days.

In S1, the control unit 11 may acquire one or more pieces of order information prescribed by the same prescription. For example, the control unit 11 may acquire two pieces of the order information with the prescription number “1” in FIG. 4.

In S2, the control unit 11 acquires medication administration record information corresponding to the order information by using one or more of the patient identification information, the date information, and the medication identification information included in the order information. Specifically, the control unit 11 acquires the medication administration record information from the medication administration record information DB 30 via the communication unit 13. The medication administration record information may be, for example, medication administration record data input by a medical staff such as a doctor or a nurse when a medication is administered to a hospitalized patient.

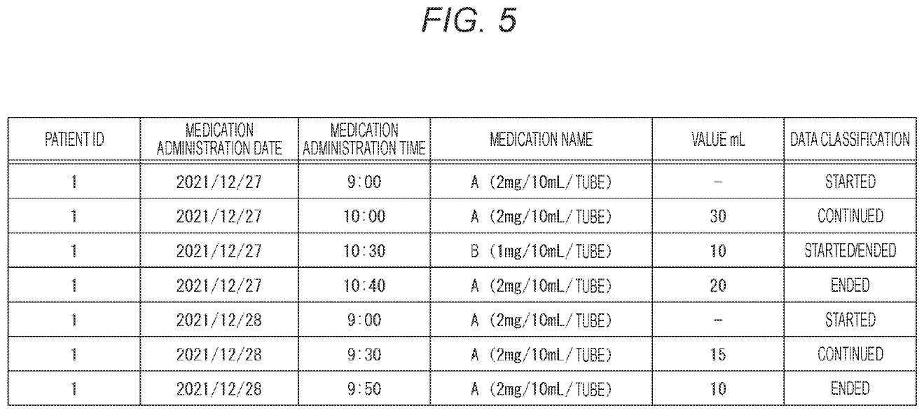

FIG. 5 is a view illustrating an example of the medication administration record information recorded in the medication administration record information DB 30 of FIG. 1. In FIG. 5, a “patient ID” is information for identifying a patient registered in the medical information system 1. In the medical information system 1 according to the present embodiment, the patient ID of the medication administration record information is indicated in the same information format as the patient ID of the order information, but may be indicated in another information format.

A “medication administration date” is information indicating a date on which a medication has been administered to the patient. A “medication administration time” is information indicating time when the medication has been administered to the patient. A “medication name” is a name of the medication prescribed to the patient. In the medical information system 1 according to the present embodiment, the medication name of the medication administration record information is indicated in the same information format as the medication name of the order information, but may be indicated in another information format.

A “value mL” is information indicating the amount (dose) of the medication that has been administered to the patient so far. A “data classification” is information indicating a type of medication administration data. For example, for a medication that is continuously administered over a certain period of time, the “data classification” may be any of “started”, “ended”, and “continued”. In addition, for example, for a medication to be administered at once, such as injection, the “data classification” may be “started/ended” (end at the same time as start).

In the example of FIG. 5, for Patient “1”, administration of “A (2 mg/10 mL/tube)” was “started” at 9:00 on Dec. 27, 2021, and “30 mL” was administered by 10:00 on the same day. Administration of “A (2 mg/10 mL/tube)” was “continued” at 10:00 on the same day and “ended” at 10:40 on the same day. “A (2 mg/10 mL/tube)” administered during a period of time from 10:00 to 10:40 on the same day is “20 mL”.

In addition, for Patient “1”, “B (1 mg/10 mL/tube)” was administered at 10:30 on Dec. 27, 2021. Since “B (1 mg/10 mL/tube)” is a medication administered at once, the data classification is “started/ended”.

Furthermore, on Dec. 28, 2021, for Patient “1”, administration of “A (2 mg/10 mL/tube)” was “started” at 9:00, and “15 mL” was administered by 9:30 on the same day. Administration of “A (2 mg/10 mL/tube)” was “continued” at 9:30 on the same day and “ended” at 9:50 on the same day. “A (2 mg/10 mL/tube)” administered during a period of time from 9:30 to 9:50 on the same day is “10 mL”.

The medication administration record information may include information other than the information illustrated in FIG. 5. For example, the medication administration record information may further include information such as an administration method of the medication, a prescription unit of the medication, a rate of administration (for example, a dose per unit time), an integrated value of doses (for example, a total amount of the administered medication or doses), remarks, and comments. For example, the administration method of the medication may include “administration route information” and “administration time information” as “administration method information”. Here, the “administration route information” may include, for example, “intraarterial injection (IA)”, “continuous intraarterial infusion (IACI)”, “intraarterial drip infusion (IADI)”, “intramuscular injection (IM)”, “intraperitoneal injection (IP)”, “intrathecal injection (IT)”, “intravenous injection (IV)”, “continuous intravenous infusion (IVCI)”, “intravenous drip infusion (IVDI)”, “central venous injection (CV)”, “central venous drip injection (CVDI)”, “oral administration (PO)”, “subcutaneous injection (SC)”, “intradermal injection (ID)”, “continuous subcutaneous injection (SCCI)”, and the like. The “administration time information” may indicate a period of time in units such as “hours” and “minutes”, for example. The configuration of the “administration method information” described herein is an example, and the present disclosure is not limited to the “administration method information” disclosed.

As can be seen by comparing FIG. 4 and FIG. 5, the order information and the medication administration record information each include information that is not included in the other. For example, the order information includes information on a medication prescribed by a doctor, but does not include information on when and how much the medication has been administered to a patient. The medication administration record information includes information on a medication administered to the patient, but there is no record of administration of “physiological saline” (or saline) in the example of FIG. 5. Even if a trained model is generated by performing machine learning using a data set for learning including a set of one of the order information and the medication administration record information and information such as patient vital data, there may be a limit to the accuracy of prediction of a prognosis of a patient acquired as a result. In addition, even if one of the order information and the medication administration record information is statistically analyzed, there may be a limit to the usefulness or reliability of information thus acquired.

The information processing apparatus 10 according to the present embodiment estimates medication administration record information corresponding to order information from the order information and combines the medication administration record information with the order information to generate the combination information 50. As described above, the control unit 11 may acquire one or more pieces of order information prescribed by the same prescription. In S2, the control unit 11 may acquire medication administration record information for each of the one or more pieces of order information acquired in S1 using one or more of patient identification information, date information, and medication identification information included in the order information. For example, for each of the one or more pieces of order information acquired in S1, the control unit 11 may acquire, as the corresponding medication administration record information, the medication administration record information including the same information as the one or more of the patient identification information, the date information, and the medication identification information included in the order information.

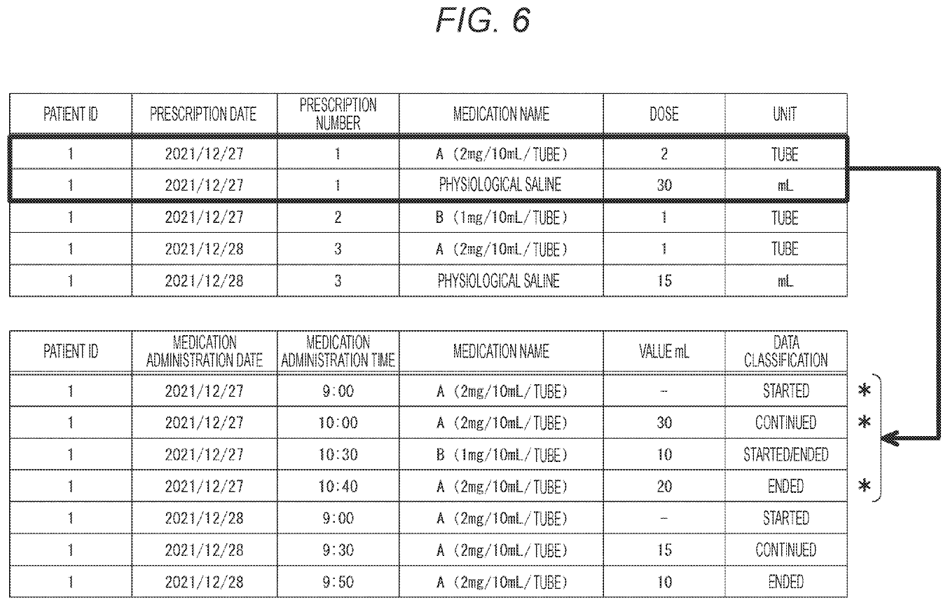

FIG. 6 is a view illustrating an example of estimating medication administration record information corresponding to order information. For example, the control unit 11 can acquire two pieces of order information with a prescription number “1” in S1. In this case, among the acquired pieces of order information, order information with a medication name “A (2 mg/10 mL/tube)” has the same patient identification information (patient ID “1”), date information (date “2021/12/27”), and medication identification information (medication name “A (2 mg/10 mL/tube)”) as three pieces of medication administration record information attached with “*” in FIG. 6. Therefore, the control unit 11 acquires the three pieces of medication administration record information attached with “*” in FIG. 6 as information corresponding to the two pieces of order information with the prescription number of “1” (medication names “A (2 mg/10 mL/tube)” and “physiological saline” (saline)).

Note that a medication amount indicated by the two pieces of order information with the prescription number “1” is 50 mL (=10 mL×2+30 mL) in total with 2 tubes of A (2 mg/10 mL) and 30 mL of physiological saline (saline). Then, a medication amount indicated by the three pieces of medication administration record information attached with “*” in FIG. 6 is 50 mL (=30 mL+20 mL). The control unit 11 may also estimate medication administration record information corresponding to order information on the basis of the fact that the amount of prescribed medications and the amount of administered medications are consistent with each other. For example, in a case where the amount of administered medications exceeds the amount of prescribed medications, the control unit 11 may determine that the medication administration record information does not correspond to the order information.

In addition, the control unit 11 may estimate, as medication administration record information corresponding to order information, medication administration record information in which administration has been recorded during a period until the predetermined number of days (for example, 1 day, 2 days, 3 days, and the like) elapses since a date indicated by date information of the order information. In addition, the control unit 11 may determine that medication administration record information for which administration was “started” on a day before the date information of the order information does not correspond to the order information.

In addition, for example, the control unit 11 may estimate medication administration record information corresponding to order information from medication administration record information with a smaller prescription number. In addition, the control unit 11 may preferentially estimate, as medication administration record information corresponding to order information, medication administration record information whose medication administration time is earlier as the medication administration record information corresponding to the order information. In this manner, in a case where there are a plurality of pieces of order information on the same day for the same medication, the control unit 11 can appropriately associate the order information with medication administration record information.

In addition, in a case where both order information and medication administration record information include medication method information indicating administration methods of medications, the control unit 11 may estimate medication administration record information in which administration by the same administration method as the administration method information of order information is recorded as the medication administration record information corresponding to the order information.

In S3, the control unit 11 adds data included in the medication administration record information acquired in S2 to the order information acquired in S1 to generate the combination information 50. For example, in a case where one or more pieces of order information prescribed by the same prescription are acquired in S1, the control unit 11 may generate, as the combination information 50, information obtained by adding data of medication administration record information acquired for each of the one or more pieces of order information to the order information. For example, the control unit 11 may generate the combination information 50 by adding a medication administration time that is time at which a medication has been administered, start and end times which are a start time and an end time of the administration of the medication, and a dose of the medication included in the medication administration record information to the order information.

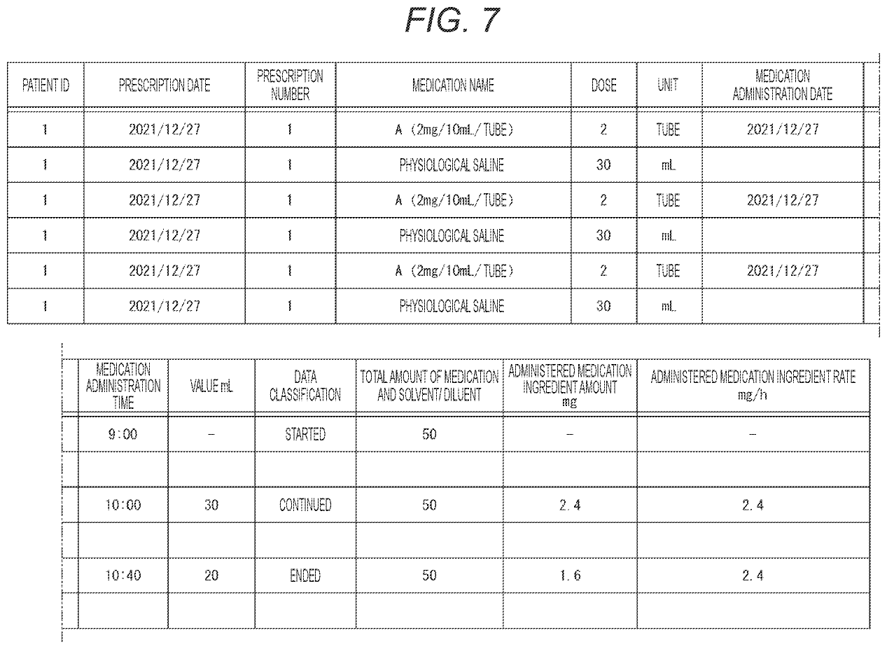

FIG. 7 is a diagram illustrating an example of the combination information 50 generated based on the order information of FIG. 4 and the medication administration record information of FIG. 5. FIG. 7 illustrates an example of the combination information 50 generated on the basis of the pieces of order information with the prescription number “1” in FIG. 6 and the three pieces of medication administration record information with “*” estimated to correspond thereto. In FIG. 7, a “patient ID”, a “prescription date”, a “prescription number”, a “medication name”, a “dose”, and a “unit” are information derived from the order information of FIG. 4. A “medication administration date”, a “medication administration time”, a “value mL”, and a “data classification” are information derived from the medication administration record information. A “total amount of medication and solvent/diluent”, a “administered medication ingredient amount mg”, and a “administered medication ingredient rate mg/h” are information that can be newly calculated as results of combining the medication administration record information with the order information.

In a case where a new item can be calculated as a result of combining the medication administration record information with the order information in this manner, the control unit 11 may include such a calculation result in the combination information 50.

In S4, the control unit 11 outputs the combination information 50 generated in S3. Specifically, for example, the control unit 11 may output the combination information 50 to the storage unit 12 for storage, to the output unit 15 for display, or to an external device via the communication unit 13. When the process in S4 is completed, the control unit 11 ends the processing of the flowchart.

As described above, the control unit 11 of the information processing apparatus 10 acquires order information including patient identification information for identifying a patient registered in the medical information system 1, date information indicating a date on which a medication has been prescribed to the patient, and medication identification information for identifying the medication prescribed to the patient. The control unit 11 uses one or more of the patient identification information, the date information, and the medication identification information to acquire medication administration record information indicating a record of a medication actually administered to the patient. The control unit 11 adds data included in the acquired medication administration record information to the order information to generate the combination information 50, and outputs the generated combination information 50.

In this manner, the information processing apparatus 10 generates and outputs the combination information 50 having more detailed information regarding the medication administered to the patient than each single piece of information of the order information and the medication administration record information acquired by the existing medical information system. Therefore, the information processing apparatus 10 enables acquisition of the more detailed information regarding the medication administered to the patient in the existing medical information system. For example, when machine learning is performed using the combination information 50, vital data, and the like to generate a trained model, it is possible to predict how the vital data and the like change in response to administration of the medication with high accuracy. Such a trained model enables appropriate prediction of a prognosis of the patient in a strained medical site such as an intensive care unit (ICU) and effective assistance when a doctor makes a treatment plan.

In addition, the control unit 11 may acquire one or more pieces of order information prescribed by the same prescription. For each of the one or more pieces of order information, the control unit 11 may acquire medication administration record information using one or more of patient identification information, date information, and medication identification information included in the order information. The control unit 11 may generate, as the combination information 50, information obtained by adding data of the medication administration record information acquired for each of the one or more pieces of order information to each of the one or more pieces of order information. In this manner, it is possible to appropriately associate the order information and the medication administration record information by collectively handling pieces of the order information prescribed by the same prescription identified with the same “prescription number” or the like.

Furthermore, the control unit 11 may acquire medication administration record information for the same patient as the patient identified by the patient identification information of the order information. Alternatively, the control unit 11 may acquire medication administration record information in which administration of the same medication as the medication identified by the medication identification information of the order information is recorded. Alternatively, the control unit 11 may acquire the medication administration record information in which the administration has been recorded during a period until a predetermined number of days elapses since the date indicated by the date information of the order information. Alternatively, the control unit 11 may acquire order information further including administration method information indicating an administration method of a medication, and acquire medication administration record information in which administration by the same administration method as the administration method indicated by the administration method information of the order information is recorded. In this manner, the control unit 11 can appropriately associate the order information with the medication administration record information by using the patient identification information, the medication identification information, the date information, the administration method information, and the like.

In addition, the control unit 11 may acquire, as the medication administration record information, information including a medication administration time that is time at which the medication has been administered, and add the medication administration time as the data to the order information to generate the combination information 50. Alternatively, the control unit 11 may acquire, as the medication administration record information, information including start and end times which are a start time and an end time of administration of the medication, and add the start and end times as the data to the order information to generate the combination information 50. Alternatively, the control unit 11 may acquire information including a dose of the medication as the medication administration record information and add the dose as the data to the order information to generate the combination information 50. In this manner, the control unit 11 can output the combination information 50 including more useful information by adding the information such as the medication administration time, the start and end times, and the dose to the order information to generate the combination information 50.

In addition, in a case where a new item, such as a total amount of the medication and a diluent, an administered medication ingredient amount, or an administered medication ingredient rate, can be calculated as a result of combining the medication administration record information with the order information, the control unit 11 may include the calculation result in the combination information 50. When such a newly calculable item is included in the combination information 50, it is possible to output the combination information 50 including more useful information.

The present disclosure is not limited to the above-described embodiment. For example, a plurality of blocks described in a block diagram may be integrated, or one block may be divided. Instead of being executed in a time-series manner according to the description, the plurality of steps described in the flowchart may be executed in parallel or in a different order, depending on the processing capacity of an apparatus performing each of the steps or as needed. In addition, modifications can be made within a scope not departing from the gist of the present disclosure.

The detailed description above describes embodiments of an information processing apparatus, an information processing method, and a program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A non-transitory computer-readable medium storing a computer program executed by a computer processor that executes a process comprising:
acquiring order information including patient identification information for identifying a patient registered in a medical information system, date information indicating a date on which a medication has been prescribed to the patient, and medication identification information for identifying the medication prescribed to the patient;
acquiring medication administration record information indicating a record of a medication administered to the patient by using one or more of the patient identification information, the date information, and the medication identification information;
adding data included in the acquired medication administration record information to the order information to generate combination information; and
outputting the generated combination information.

2. The computer-readable medium according to claim 1, further comprising:
acquiring one or more pieces of the order information prescribed by a prescription slip, the prescription slip having order information that includes the same order information as another prescription slip for the patient;
for each of the one or more pieces of order information, acquiring the medication administration record information using one or more of the patient identification information, the date information, and the medication identification information included in the order information; and
generating information obtained by adding data of the medication administration record information acquired for each of the one or more pieces of order information to each of the one or more pieces of order information as the combination information.

3. The computer-readable medium according to claim 1, further comprising:
acquiring the medication administration record information on the patient identified by the patient identification information of the order information.

4. The computer-readable medium according to claim 1, further comprising:
acquiring the medication administration record information in which administration of the medication identified by the medication identification information of the order information that has been recorded.

5. The computer-readable medium according to claim 1, further comprising:
acquiring the medication administration record information in which administration has been recorded during a period until a predetermined number of days elapses since the date indicated by the date information of the order information.

6. The computer-readable medium according to claim 1, further comprising:
acquiring the order information further including administration method information indicating an administration method of the medication; and
acquiring the medication administration record information in which administration by an administration method indicated by the administration method information of the order information.

7. The computer-readable medium according to claim 1, further comprising:
acquiring information including a medication administration time that is time at which the medication has been administered as the medication administration record information; and
adding the medication administration time as the data to the order information to generate the combination information.

8. The computer-readable medium according to claim 1, further comprising:
acquiring information including start and end times which are a start time and an end time of administration of the medication as the medication administration record information; and
adding the start and end times as the data to the order information to generate the combination information.

9. The computer-readable medium according to claim 1, further comprising:
acquiring information including a dose of the medication as the medication administration record information; and
adding the dose as the data to the order information to generate the combination information.

10. An information processing apparatus comprising:
a control unit configured to:
acquire order information including patient identification information for identifying a patient registered in a medical information system, date information indicating a date on which a medication has been prescribed to the patient, and medication identification information for identifying the medication prescribed to the patient;
acquire medication administration record information indicating a record of a medication administered to the patient by using one or more of the patient identification information, the date information, and the medication identification information;
add data included in the acquired medication administration record information to the order information to generate combination information; and
output the generated combination information.

11. The information processing apparatus according to claim 10, wherein the control unit is configured to:
acquire one or more pieces of the order information prescribed by a prescription slip, the prescription slip having order information that includes the same order information as another prescription slip for the patient;
for each of the one or more pieces of order information, acquire the medication administration record information using one or more of the patient identification information, the date information, and the medication identification information included in the order information; and
generate information obtained by adding data of the medication administration record information acquired for each of the one or more pieces of order information to each of the one or more pieces of order information as the combination information.

12. An information processing method executed by a computer, the information processing method comprising:
acquiring order information including patient identification information for identifying a patient registered in a medical information system, date information indicating a date on which a medication has been prescribed to the patient, and medication identification information for identifying the medication prescribed to the patient;
acquiring medication administration record information indicating a record of a medication administered to the patient by using one or more of the patient identification information, the date information, and the medication identification information;
adding data included in the acquired medication administration record information to the order information to generate combination information; and
outputting the generated combination information.

13. The information processing method according to claim 12, further comprising:
acquiring one or more pieces of the order information prescribed by a prescription slip, the prescription slip having order information that includes the same order information as another prescription slip for the patient;
for each of the one or more pieces of order information, acquiring the medication administration record information using one or more of the patient identification information, the date information, and the medication identification information included in the order information; and
generating information obtained by adding data of the medication administration record information acquired for each of the one or more pieces of order information to each of the one or more pieces of order information as the combination information.

14. The information processing method according to claim 12, further comprising:
acquiring the medication administration record information on the patient identified by the patient identification information of the order information.

15. The information processing method according to claim 12, further acquiring the medication administration record information in which administration of the medication identified by the medication identification information of the order information that has been recorded.

16. The information processing method according to claim 12, further comprising:
acquiring the medication administration record information in which administration has been recorded during a period until a predetermined number of days elapses since the date indicated by the date information of the order information.

17. The information processing method according to claim 12, further comprising:
acquiring the order information further including administration method information indicating an administration method of the medication; and
acquiring the medication administration record information in which administration by an administration method indicated by the administration method information of the order information.

18. The information processing method according to claim 12, further comprising:
acquiring information including a medication administration time that is time at which the medication has been administered as the medication administration record information: and
adding the medication administration time as the data to the order information to generate the combination information.

19. The information processing method according to claim 12, further comprising:
acquiring information including start and end times which are a start time and an end time of administration of the medication as the medication administration record information; and
adding the start and end times as the data to the order information to generate the combination information.

20. The information processing method according to claim 12, further comprising:
acquiring information including a dose of the medication as the medication administration record information; and
adding the dose as the data to the order information to generate the combination information.
